(19) United States
(12) Patent Application Publication
(10) Pub. No.: US 2005/0019318 A1
(43) Pub. Date: Jan. 27, 2005

(54) Methods and compositions for prevention of angioproliferation

(76) Inventors: Emil V. Kozarov, Miramar, FL (US); Ann Progulske-Fox, Keystone Heights, FL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/477,007

(22) PCT Filed: May 2, 2002
(86) PCT No.: PCT/US02/13590

Related U.S. Application Data
(63) Continuation of application No. 09/849,115, filed on May 5, 2001.

(51) Int. Cl. A61K 38/48; C12N 9/52

(52) U.S. Cl. 424/94.63; 435/220

(57) ABSTRACT

The invention provides pharmaceutical compositions comprising Porphyromonas gingivalis protease and hemagglutinin polypeptides that have anti-angiogenic activity and methods for their use.

### PRIORITY

This application claims priority to U.S. Ser. No. 09/849,115, filed on May 5, 2001, which is incorporated by reference in its entirety herein

### FIELD OF THE INVENTION

The invention provides compositions and methods for the treatment, prevention and amelioration of angioproliferative conditions.

### BACKGROUND INFORMATION

Cancer is the second leading cause of death in the United States, accounting for over one half million deaths per year. (National Vital Statistics Report, 1998, Vol. 48, No. 11). The total economic cost associated with cancer has been estimated to be over $100 billion dollars annually. (Brown, M. L. et al. In Cancer Epidemiology and Prevention, 1996). There is currently no cure for the disease, but several lines of research appear promising. Of these, research directed at preventing angiogenesis offers the most hope.

Angiogenesis involves a complex biochemical cascade of events that leads to new blood vessel formation in, for example, developing tumors. For cells to survive, each must have some communication, direct or indirect with the existing vasculature in order to obtain nutrients and oxygen, and to offload metabolic waste products. Active vascularization is normally observed following injury to a tissue, during development, or in response to ovulation in females. However, abnormal rapid proliferation of blood vessels is also observed in areas where cancerous masses have developed. Because the rate of cell mass growth is limited by the degree of vascularization present, cancer cells release chemical substances into the surrounding environment to induce nearby, nourishing blood vessels to grow toward the proliferating cancer cell mass. For example, angiopoietin-1 basic fibroblast growth factor (bFGF), vascular endothelial growth factor (VEGF) and other substances are released from cancer cells to coax surrounding blood vessels to grow collateral vessels toward the tumor. Once vascularized, a tumor mass may grow locally, or may metastasize and begin to spread through the bloodstream and lymphatic system to other parts of the body, causing significant damage. For example, vascularization of the retina in diabetic retinopathy can lead to retinal detachment resulting in blindness. Metastasis is a hallmark for malignancy, which in extreme cases may lead to rapid death of an individual. Ironically, some tumors may also secrete angiostatic substances to inhibit tumor growth (Chen et al., Cancer Res. 1995, 55, 4230-4233; O'Reilly, Cell 1997, Jan. 24 (88):277-285). Thus, it appears that in healthy individuals angiogenesis associated with tumor growth may be regulated by a fine balance between the release of angiogenic factors and the release of angiostatic factors. It is believed that by blocking the process of angiogenesis, tumor growth can be suspended, which in turn would lead to cancer remission.

Most early research directed at preventing angiogenesis involved exposing various cell lines to angiostatic compounds and assessing the degree of proliferation either in vivo or in vitro. The National Cancer Institute, for example, uses proliferation, migration and cord formation assays in HUVE cells for its anti-angiogenesis testing. Several angiostatic agents that function to prevent the proliferation of cancer cells have been isolated and tested. For example, administration of Angiostatin has been shown to suppress vascular endothelial cell proliferation, thereby reducing the size and lethality of tumors (Folman J., Forum Genova 1999 July-Dec. 9 (3 Suppl. 3): 5962). Recombinant Endostatin (baculovirus) has been used to inhibit the proliferation of bovine capillary endothelial cells. (O'Reilly, et al., Cell, 1994, Oct. 21; 79(2):185-8). Until recently, angiostatic compounds have included only those substances capable of preventing proliferation of cells. However, a growing body of evidence demonstrates that agents which inhibit proliferation via cellular detachment from tumor masses perform an analogous function.

Recent studies have been directed at interfering or disrupting the mechanisms involved in cell-cell or cell-matrix binding as a means to reduce or eliminate cancerous growth. Cells will not proliferate if they are not first attached to a surface. For example, impaired cell-matrix contact leads to anoikis (epithelial apoptosis) (Vitale et al., FEBS Lett. 1999 462 (1-2:57-60); Attwell et al., Oncogene 2000 19(33):3811-5; Rosen, J. Cell. Biol 2000 149(2):447-56); Rytomaa et al., Curr. Biol. 1999 9(18):1043-6 or endothelial apoptosis (Erdreich-Epstein, Cancer Res. 2000 Feb. 1; 60(3):71221). This anchorage dependence is mediated, in part, by cell surface molecules known as integrins. See e.g., Erdreich-Epstein et al., Cancer Res., 2000 Feb. 1; 60(3):712-21; Lee & Juliano, Mol. Biol. Cell, 2000 Jun. 11(6):1973-87; Kawahara, J. Cancer Res. Clin. Oncol, 1995 1212 (3):133-40; Lee, Mol. Biol. Cell 2000 11(6):1973-87; Ruoslahti, Kidney Int. 1997 51(5):1413-7; Brassard et al., Exp. Cell. Res. 1999 251 (1):33-45; Kottke et al., J. Biol. Chem. 1999 274(22):15927-36.

Anti-angiogenic approaches are the most recent and promising avenue in cancer treatment. Agents capable of blocking vascularization of neoplastic tissue can prevent subsequent growth of transformed tissue and can lead to existing tissue remission. Anti-angiogenic activity has been detected for several endogenous factors. For example, combrestatin A-4 disodium phosphate (CA4DP) (Dark, Cancer Res. 1997 May 15; 57 (10):1829-34), a purified human PSA compound (WIPO PCT publication No. WO 99/60984), Endostatin (add, et al., Biochem. Biophys. Res. Commun. 1999 Sep. 24:263(2):3405), so (AIbini, Faseb J. 1999 13(6):647-55), Epidermal Growth Factors (EGF) (Kotke, et al., J. Bio. Cam 1999 May 28;274(22):15927-36), and angiostatin (Stack, Biochem J., 1999 340 (pt1):77-84) have anti-angiogenic activity.

For many years, scientists have been in search of therapeutics that can be used to prevent periodontal diseases, including gum infections and tooth decay. One organism that has been identified as a potential etiologic agent of such pathologies as gingivitis and periodontal disease is the pathogen Porphyromonas gingivalis. Sequences from the pathogen have been cloned and sequenced. Examples of such work can be found in U.S. Pat. Nos. 5,824,791 and 5,830,710, both of which are hereby incorporated herein by reference in their entity.

Proteolytic enzymes of Porphyromonas gingivalis are the main tool for providing nutrients to these asaccharolytic bacteria and are also important virulence factors. These enzymes have been shown to degrade basement membrane matrix proteins (Uitto, Oral Microbiology and Immunology 1988 3:97-102), (Smalley, Arch. Oral Biol. 1988 33 (5):323-9) and purified P. gingivalis cysteine protease has been shown to disrupt the basement membrane of human carcinoma monolayer (Shah, J. Periodontol. 1992 63(1):44-51).

Targeted disruption of fibronectin-integrin interactions in human gingival fibroblasts has been demonstrated by the RI protease of P. gingivalis W50 (Scragg, et al., Infect. Immun. 1999 67(4):1837-43). Other studies have examined the function of P. gingivalis extract on cell-cell and cell-matrix bonds. Cell detachment from each other and from the underlying surface correlates with the cysteine-dependent proteolytic activity of P. gingivalis (Johansson, Eur. J. Oral. Sci. 1998 106(4):863-71). B1-integrin, occluding and E-cadherin are targeted by P. gingivalis proteolytic activity in canine epithelial cells (Katz, et al., Infect. Immun. 2000 68(3):1441-9).

### SUMMARY OF THE INVENTION

It is an object of the invention to provide compositions and methods for treatment, prevention or amelioration of angioproliferitive conditions. This and other objects of the invention are provided by one or more of the embodiments described below.

One embodiment of the invention provides a composition for the t ent, prevention, or amelioration of an angioproliferitive condition. The composition comprises a pharmaceutically effective amount of a substantially purified Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof, wherein the polypeptide has anti-angiogenic activity, and a pharmaceutically acceptable excipient. The polypeptide can be selected from the group consisting of rgpA, rgpB, kgp, hag, prtT and tla polypeptides. The polypeptide can have at least about 90% sequence identity with a rgpA, rgpB, kgp, hag, prtT, or tla polypeptide and has anti-angiogenic activity. The polypeptide can be a biologically functional homolog, or isoform of a rgpA, rgpB, kgp, hag, prtT or tla polypeptide. The polypeptide can be a fragment of a Porphyromonas gingivalis arginine specific cysteine protease polypeptide, Porphyromonas gingivalis lysine specific cystine protease polypeptide, or Porphyromonas gingivalis hemagglutinin polypeptide, such as HA2. Such a fragment has anti-angiogenic activity.

An angioproliferative condition can be carcinoma, sarcoma, melanoma, benign tumor, ocular retinopathy, retrolental fibroplasias, psoriasis, angiofibromas, endometriosis, hemangioma, rheumatoid arthritis, Osler Webber Syndrome, myocardial angiogenesis, telangiectasia, hemophiliac joints, wound granulation, intestinal adhesions, post-surgery adhesions, arteriosclerosis, scleroderma, hypertrophic scars, cat scratch disease, Helicobacter pylori ulcers, capillary proliferation within atherosclerotic plaque, or a combination thereof.

Another embodiment of the invention provides a method for the treatment, prevention, or amelioration of an angioproliferative condition. The method comprises administering a pharmaceutically effective amount of a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof to a patient in need thereof, wherein the polypeptide has anti-angiogenic activity, whereby the angioproliferative condition is treated, prevented or ameliorated. The method can comprise contacting a vasculature supplying a biological structure affected by the angioproliferative condition with the polypeptide. The polypeptide can be contacted with a basolateral surface of the vasculature.

Yet another embodiment of the invention provides a method for potentiating effects of a chemotherapeutically effective agent. The method comprises administering a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof, wherein the polypeptide has disrupts cell-cell adhesion, cell-matrix adhesion, or both; and a chemotherapeutically effective agent to a patient. Effects of the chemotherapeutically effective agent arm potentiated.

Still another embodiment of the invention provides a method for preventing the formation of now vasculature required for implantation or sustenance of a fertilized mammalian ovum. The method comprises administering a Porphyromonas gingivalis amine specific cysteine protease polypeptide, Porphyromonas gingivalis lysine specific cysteine protease polypeptide, Porphyromonas gingivalis hemagglutinin polypeptide, or a combination thereof to a mammal, wherein the polypeptide has anti-angiogenic activity, whereby the formation of new vasculature required for implantation or sustenance of a fertilized mammalian ovum is prevented.

Even another embodiment of the invention provides a pharmaceutical composition for facilitating passage of compounds through a blood-brain barrier comprising a pharmaceutically effective amount of a substantially purified Porphyromonas gingivalis arginine specific cysteine protease polypeptide, Porphyromonas gingivalis lysine specific cysteine protease polypeptide, a Porphyromonas gingivalis hemagglutinin polypeptide, or a combination thereof, and a pharmaceutically acceptable excipient. The pharmaceutical composition can further include a compound to be passed through the blood-brain barrier.

Another embodiment of the invention provides a method of delivering a compound through a blood-brain barrier of a patient comprising administering a pharmaceutically effective amount of a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof and the compound, whereby the compound is passed through the blood-brain barrier.

Still another embodiment of the invention provides a method of degrading a tumor. The method comprises administering a pharmaceutically effective amount of a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof to a patient having a tumor, wherein the polypeptide degrades cell-cell bonds, cell-matrix bonds, or both cell-cell bonds and cell-matrix bonds of the tumor, whereby the tumor is degraded.

Therefore, the present invention discloses anti-angiogenic compositions of mater and methods of their use, which are capable of disrupting endothelial tissue growth and proliferation. The methods involve local or systemic application of a P. gingivalis polypeptide to a targeted tissue. The disclosed polypeptides can inhibit angiogenesis associated with malignant tumor proliferation by, for example, disrupting endothelial layer cell-cell and cell-matrix adhesion bonds. Use of these compounds and methods have advantages over conventional treatments such as chemotherapy, because it targets only growing vessels, while leaving intact vessels unaffected.

The present invention provides a novel strategy that uses protein, peptide and nucleic acid sequences of Porphyromonas gingivalis to treat or prevent angioproliferative conditions.

### BRIEF DESCRIPTION OF THE FIGURES

FIG. 1 demonstrates percent detachment of active Human Umbilical Vein Endothelial Cells (HUVEC) after 24 hours of treatment with a protein extract from P. gingivalis. Represented are mean values from triplicate experiments.

FIG. 2 demonstrates percent detachment of active HUVEC after 48 hours of treatment with a protein extract from P. gingivalis.

FIG. 3 demonstrates percent detachment of quiescent HUVEC after 24 hours of treatment with a protein extract from P. gingivalis.

FIG. 4 demonstrates percent detachment of quiescent HUVEC after 48 hours of treatment with a protein extract from P. gingivalis.

FIG. 5 demonstrates percent detachment of active human non-small cell lung carcinoma cell line (A549) after 24 hous of treatment with a protein extract from P. gingivalis.

FIG. 6 demonstrates percent detachment of active human non-small cell lung carcinoma cell line (A549) after 48 hours of treatment with protein extract from P. gingivalis.

FIG. 7 demonstrates percent detachment of quiescent human non-small cell lung carcinoma cell line (A549) after 24 hours of treatment with a protein extract from P. gingivalis.

FIG. 8 demonstrates percent detachment of quiescent human non-small cell lung carcinoma cell line (A549) after 48 hours of treatment with a protein extract from P. gingivalis.

FIG. 9 demonstrates the reduction in human vascular endothelial cell migration after exposure to a protein extract from P. gingivalis over a 24 hour period.

FIG. 10 demonstrates detachment of human non-small cell lung carcinoma cell line (A549) after treatment with a proteinase extract of P. gingivalis alone and with an extract of Bacteroides fragilis expressing PrtP protease from P. gingivalis.

FIG. 11 is a table showing the degree of proliferation inhibition of a HUH7 cell line exposed to P. gingivalis and E. coli extract, and to P. gingivalis cells in the presence or absence of inhibitors.

FIG. 12A-E shows the results of reactivity of a HUH7 cell line with anti-occludin antibodies. FIG. 12A depicts non-treated control HUH7 cells. FIG. 12B depicts the HUH7 cells treated with P. gingivalis extract. FIG. 12C depicts HUH7 cells treated with P. gingivalis extract in the presence of the inhibitor TLCK. FIG. 12D depicts results of treatment with heat-treated P. gingivalis extract. FIG. 12E depicts HUH7 cells treated with a control E. coli extract; the occludin network is intact.

FIG. 13A-D show the result of reactivity of a HUH7 cell tine with an anti-pan cadherin antibody. FIG. 13A depicts non-treated control HUH7 cells. FIG. 13B depicts HUH7 cells treated with P. gingivalis extract. FIG. 13C depicts HUH7 cells treated with P. gingivalis extract in the presence of inhibitor TLCK. FIG. 13D depicts HUH7 cells treated with heat inactivated P. gingivalis extract.

FIG. 14A-C shows proliferation inhibition of a HUVEC polarized cell line. FIG. 14A depicts non-treated, control polarized human endothelial cell layer, ECV-304. FIG. 14B shows ECV-304 cells treated basolaterally (lower chamber) with a 60% fraction of P. gingivalis culture liquid proteins. FIG. 14C depicts the results of ECV-304 cells lumenally treated with a 60% fraction of P. gingivalis culture liquid proteins.

FIG. 15 demonstrates total cell number reduction as result of treatment with P. gingivalis strain W83 extract.

FIG. 16 demonstrates that detachment of HUVE cells is reduced by inhibitors or heating of SPF.

FIG. 17 demonstrates transendothelial resistance of HUVE cells upon treatment with P. gingivalis soluble protein fraction (SPF) over a period of 6 hours.

FIG. 18 demonstrates transendothelial resistance of HUVE cells upon treatment with P. gingivalis soluble protein fraction (SPF) over a period of 8 days.

FIG. 19A-C shows contrast micrographs of HUVE cell layers that were treated with P. gingivalis SPF or mock-treated at day 8. FIG. 19A shows treated cells. FIG. 19B shows BL-treated cells. FIG. 19C shows mock-treated control cells.

FIG. 20A-C show contrast micrographs of HUVE cells treated with extracts of E. coli. FIG. 20A shows HUVE cells treated with control E. coli host extract. FIG. 20B shows control mock-treated cells. FIG. 20C shows cells treated with extracts of E. coli host cells that express a 15 kDa internal fragment of a P. gingivalis HagA repeat.

FIG. 21 shows proliferation inhibition of HUVE cells treated with HA2 15-kDa P. gingivalis protein cloned in E. coli. (E. coli 0.5: control E. coli protein extract at 0.5 mg/ml. E. coli HA2 0.5: E. coli protein extract containing HA2, the 15-kDa polypeptide at 0.5 mg/ml.

FIG. 22 shows potential pathways for leakage from blood vessels in tumors. Intercellular openings (arrows) between lining cells of a murine tumor vessel viewed by scanning EM. FIGS. 22A and B: Multiple large intercellular openings (arrow, FIG. 22A) and three smaller transcellular holes (arrows, FIG. 22B) in branched lining cells of a tumor vessel. The intercellular openings are much larger than the holes. The boxed region in FIG. 22A is shown at higher magnification in FIG. 22B.

### DETAILED DESCRIPTION OF THE INVENTION

“Angiogenesis” or “angioproliferation” means conditions of rapid development of vascular supply to a particular organ or biological site. The rapid development can be uncontrolled and/or pathogenic, as in the development of a tumor. However, angiogenisis or angioproliferation can also be associated with non-pathological conditions such as angiogenesis which occurs upon implantation of a fertilized ova. An anti-angiogenic or anti-angioproliferative composition is capable of reducing or preventing angiogenisis or angioproliferation.

The present invention is directed to arginine- or lysine-specific cysteine protease polypeptides and hemagglutinin polypeptides derived from P. gingivalis. The polypeptides can be used to treat or prevent angioproliferative conditions, including but not limited to melanoma, sarcoma, and carcinomas including, for example, breast, colon, lung and prostate carcinomas. Additional pathologies susceptible to treatment according to the present invention include benign tumor, ocular retinopathy, retrolental fibroplasias, psoriasis, angiofibromas, endometriosis, hemangioma, rheumatoid arthritis, Osler Webber Syndrome, myocardial angiogenesis, telangiectasia, hemophiliac joints, wound granulation, intestinal adhesions, post-surgery adhesions, scleroderma, hypertrophic scars, cat scratch disease, and Helicobacter pylori ulcer and capillary proliferation within atherosclerotic plaque. Post-surgery adhesions are common complication of gynecologic and abdominal surgery. Such complications can lead to infertility, ectopic pregnancy, chronic pain, prolonged recovery and intestinal obstruction. Adhesions following surgery can be prevented by applying polypeptides of the invention at the end of the operation.

Furthermore, the compositions of the invention can be used to control non-pathogenic angiogenic conditions, such as using the compositions as a contraceptive to prevent implantation of fertilized ova.

Polypeptides of the Invention

Compositions of the invention comprise a substantially purified Porphymonas gingivalis arginine-specific cysteine protease polypeptide, a Porphymonas gingivalis lysine-specific cysteine protease polypeptide, a Porphymonas gingivalis hemagglutinin polypeptide or combinations thereof. These polypeptides are expressed from members of the P. gingivalis protease and hemagglutinin gene family. See e.g., Curtis et al., J. Periodont. Res. 34:494 (1999). “Substantially purified” refers to polypeptides that are removed from their natural environment, isolated or separated, and are at least 60% free, preferably 75% free, and most preferably 90% free from other components with which they are naturally associated.

Polypeptides of the invention include rgpA arginine-specific cysteine proteases. P. gingivalis rgpA proteases are a group of proteases produced by genes of a homologous loci in different of P. gingivalis. Examples of the rgpA proteases are: prpR1 (Accession number X82680), rgp-1 (Accession number U15282), rgpA (Accession number D26470), prtR (Accession number L26341), prtH (Accession number L27483), and hagE (Accession number AF026946), which is similar to rgp (Accession number A55426). Isoforms of rgpA proteases are also part of the invention and include, for example, RgpA_{(cat)}, mt-RgpA_{(cat)} and HRgpA isoforms. See, e.g., Curtis et al., J. Periodont. Res. 34:494 (1999).

P. gingivalis rgpB arginine-specific proteases are also polypeptides of the invention Examples of rgpB proteases include prtRII (Accession number AF007124), rgp-2 (Accession number U85038), rgpB (Accession number D64081), and prR2 (Rangarajan, Mol Microbiol 1997 Mar. 23(5):955-65). The rgpB proteases are all highly related and are derived from homologous genes which occur in different strains of P. gingivalis. Isoforms of rgpB proteases are also part of the invention and include, for example, RgpB and mt-RgpB isoforms. See e.g., Curtis et al., J. Periodont. Res. 34:494 (1999).

Lysine-specific proteases of P. gingivalis include kgp proteases. kgp proteases include, for example, prtK (Accession number U75366), kgp (Accession numbers U54691; D83258), prtP (Accession numbers U42210; AF017059), kgp(381)-hagD (Accession number U68468). The kgp proteases are all highly related and are derived from homologous genes in different stains of P. gingivalis. Isoforms of kgp proteases are also included in the invention.

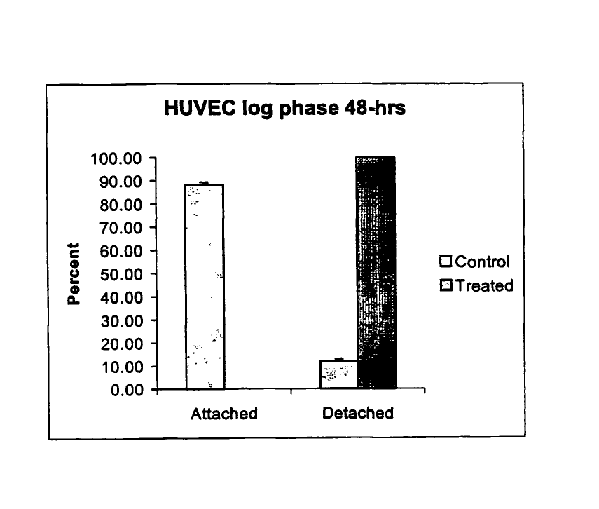

Hemagglutinin polypeptides of the invention include hagA, hagB, hagC, hagD, hagE, tla (e.g., Accession number Y07618) and prtT (Accession number S75942; M83096). See also, e.g., U.S. Pat. Nos. 5,824,791 and 5,830,710 and Aduse-Opoku et al., J. Bacteriol. 179:4778-4788 (1997). As disclosed in U.S. Pat. No. 5,824,791, a hag gene can be a 7887 bp molecule, which encodes a gene product of 2628 amino acids. Within the gene product, there are four repeat segments: HArep1, HArep2, HArep3 and HArep4. Bach of these segments contains a 15-kDa fragment, HA2, that possesses anti-angiogenic activity and is found in the secreted protein fraction. HA2 is instrumental in production of necrosis, which leads to periodontal disease. HA2 is located within a hagA sequence between amino acids 683 and 819 in the first repeat and is similarly located in the other repeats (see Han et al., Infect. Immun. Page 4002, FIG. 2, U.S. Pat. Nos. 5,824,791 and 5,830,710). P. gingivalis hemagglutinin polypeptides have cysteine protease activity against arginine containing substrates. Nishikata and Yoshimura, Biochem Bio. Phys. Res. Comm 178:336-40 (1991). P. gingivalis hemagglutinins also function as attachment factors and the substrate binding site is responsible for attachment to erythrocytes. Hemagglutinins also contain “peptidase C25 family” activity. The entire hagA, hagB, hagC, hagD, hagE, prtT or tla polypeptide can be used to produce anti-angiogenic effects. Alternatively, any of the HArep sequences or HA2 can be used Additionally, compounds with similar activity can also be developed which have equal or greater potency than polypeptides of the invention, and such compounds come within the scope of this invention. For example, one of skill in the art can design peptidomimetics using, for example, directed protein evolution or by minimizing the size of the polypeptide so that improved or smaller versions of the polypeptide are developed.

Polypeptides of the invention include polypeptides produced from any P. gingivalis rgpA, rgpB, kgp, hag, prtT or tla gene. It is well known in the art that the proteases and hemagglutinins of P. gingivalis are produced from a family of protease and hemagglutinin genes that are highly related. This gene family is known to vary from strain to strain of P. gingivalis. See e.g., Curtis et al., J. Periodont. Res. 34:494 (1999). The invention includes all polypeptides produced from the rgpA, rgpB, kgp, hag, prtT or tla gene family that have anti-angiogenic activity. One of skill in the art could determine whether a protease or hemagglutinin gene or polypeptide fell within the rgpA, rgpB, kgp, hag, prtT or tla family by comparing the nucleotide or protein sequence of the gene or protein in question to the known sequences of these genes and proteins.

Curtis et al. (J. Peridont. Res. 34:464 (1999)) describes these genes families and discloses examples of genes that fall within the P. gingivalis protease and hemagglutinin gene family. U.S. Pat. Nos. 5,824,791 and 5,830,710 further describes genes that fall within the hag family. All of these related polypeptides have cysteine protease activity and sequence homology and as such, can be classified into the protease-hemagglutinin family.

Polypeptides of the invention can either be full-length polypeptides or fragments of polypeptides. For example, fragments of polypeptides of the invention can comprise about 10, 25, 50, 100, 200, 250, 500, 750, or 1,000 amino acids of polypeptides of the invention Percent sequence identity ha an art recognized meaning and there are a no of methods to measure identity between two polypeptide or polynucleotide sequences. See, e.g., Lesk, Ed, Computational Molecule Biology, Oxford University Press, New York, (1988); Smith, Ed, Biocomputing: Informatics And Genome Projects, Academic Press, New York, (1993); Griffin & Griffin, Eds., Computer Analysis Of Sequence Data, Part I, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Mole Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. App. Math., 2:482-489 (1981)). For example, the computer program ALIGN which employs the FASTA algorithm can be used, with an affine gap search with a gap open penalty of −12 and a gap extension penalty of −2.

When using any of the sequence alignment programs to determine whether a particular sequence is, for instance, about 95% identical to a reference sequence, the parameters are set such that the percentage of identity is calculated over the full length of the reference polynucleotide and that gaps in identity of up to 5% of the total number of nucleotides in the reference polynucleotide are allowed.

Amino acid sequences having at least about 75, preferably at least about 90, 95, 96, 98, or 99% sequence identity to amino acid sequences of P. gingivalis rgpA, rgpB, kgp, hag, prtT or tla polypeptides and have anti-angiogenic activity are also polypeptides of the invention.

Also included in the invention are polypeptides having amino acid sequences which are at least about 75, preferably at least about 90, 95, 96, 98, or 99% sequence identity toamino acid sequences of P. gingivalis rgpA, rgpB, kgp, hag, prtT or tla proteins that are produced by an organism other than P. gingivalis and have anti-angiogenic activity.

Polypeptides of the invention also are biologically functional homologs, analogs and isoforms of rgpA, rgpB, kgp, hag, prtT or tla polypeptides. A biologically functional homolog is a polypeptide that has sequence identity to an analogous polypeptide from another species. Homologs can be naturally occurring or can be a polypeptide that does not occur in nature. An isoform is encoded by a distinct mRNA splice variant and can be a naturally occurring polypeptide or can be a polypeptide that does not occur in nature. An analog is a polypeptide having alterations involving one or more amino acid insertions and deletions and/or conservative amino acid substitutions. A homolog, analog or isoform of the invention comprises at least about 75, 90, 95, 96, 98 or 99% sequence identity to a rgpA, rgpB, kgp, hag, prtT or tla polypeptide. A biologically functional homolog, analog or isoform has at least one biological activity of a rgpA, rgpB, kgp, hag, prtT or tla polypeptide. That is, they are capable of preventing endothelial cell proliferation, disruption of vascular endothelium, promoting cellular detachment, inhibiting migration of endothelial cells, blocking formation of new blood vessels, or destroying existing blood vessels feeding, for example, tumors.

A biologically functional homolog or analog, isoform, or fragment can be tested for biological activity using, for example a cell proliferation/cell detachment assay (see Example 1), a migration inhibition assay (see Example 2), an occludin-stain junction assay (see Example 7), catherin-stain junction assay (see Example 8) proliferation inhibition of polarized cell assay (see Example 10), degradation of α5β1 integrin assay (see Example 11), or transendothelial resistance assay (see Example 12). A biologically functional homolog, analog, isoform or fragment has about 85%, 90%, 95%, 98%, 99%, 100%, 105%, or 110% biological activity of a specific specie in question.

In one embodiment of the invention, a polypeptide of the invention is produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. A polypeptide of the invention can also be chemically synthesized.

If desired, a polypeptide can be produced as a fusion protein, which can also contain other amino acid sequences, such as amino acid linkers or signal sequences, as well as ligands useful in protein purification, such as glutathione-S-transferase, histidine tag, and staphylococcal protein A. More than one polypeptide of the invention can be present in a fusion protein.

Biological Activity of Polypeptides of the Invention

Polypeptides of the invention have anti-angiogenic biological activity. That is, they are capable of preventing endothelial cell proliferation, disruption of vascular endothelium, promoting cellular detachment, inhibiting migration of endothelial cells, blocking formation of new blood vessels, or destroying existing blood vessels feeding, for example, tumors or combinations thereof. Therefore, compositions of the invention are capable of inhibiting of tumor enlargement, decreasing tumor mass, and inhabiting, reducing, or preventing related biological processes associated with angiogenesis and vascular supply to a particular biological organ or location, or combinations thereof. In addition, the polypeptides of the invention can be capable of disintegrating cell-to-matrix and cell-to-cell bonds in, for example, a tumor such that the tumor is reduced or eliminated. A polypeptide of the invention has at least one of the above-mentioned biological activities.

Polypeptides of the invention can act on cell surface adhesion molecules (CAM's), from the basolateral side of the endothelium. Polypeptides of the invention can inhibit angiogenesis by targeting integrin in cell-cell and cell-matrix adhesion bonds. Of particular importance is the fact that vasculature supplying tumor tissues is aberrantly leaky. Regular blood vessels are well formed and have a well-developed adhesion system to keep them together, while blood vessels supplying tumors are poorly formed and extremely leaky (Herlyn, Immunother. 1999 May, 22(3):185); see also, Hashizume, et al., Am. J. Pathol. 2000 April 156(4):1363-80). As a result, locally or systemically administered polypeptides of the invention leak out of the vasculature that supplies tumor tissue. Access to the basolateral surface of the vasculature is thereby achieved, which results in disruption of the vascular at that location such that di-ation of the tumor occurs. Therefore, compositions of the invention can disrupt angiogenesis without affecting integrity of normal blood vessels.

Methods of Treatment

Compositions of the invention can be used to treat several conditions in mammals, including humans. For example, angioproliferative conditions can be treated, prevented or ameliorated according the invention. Methods of treatment comprise administering to a patient having an angioproliferative condition a pharmaceutically effective mount of a composition of the invention such that an anti-angiogenic effect is achieved Angioproliferative conditions include, for example, carcinoma, sarcoma, melanoma, benign tumor, ocular retinopathy, retrolental fibroplasias, psoriasis, angiofibromas, endometriosis, hemangioma, rheumatoid arthritis, Osler Webber Syndrome, myocardial angiogenesis, telangiectasia, hemophiliac joints, wound granulation, intestinal adhesions, post-surgery adhesions, atherosclerosis, scleroderma, hypertrophic scars, cat scratch disease, Helicobacter pylori ulcers, capillary proliferation within atherosclerotic plaque, or a combination thereof.

An angioproliferative condition can be selectively treated according to the invention by contacting vasculature supplying a biological structure affected by an angioproliferative condition with an anti-angiogenically effective amount of a composition of the invention. Due to the known leakiness of vasculature supplying, for example, a tumor, local or systemic administration of a composition of this invention facilitates contact with the basolateral surface of said vasculature, including the endothelium, with no effect on normal tissues.

Compositions of the invention can also be used to potentiate the effects of a chemotherapeutically effective agent. Such methods comprise co-administering a chemotherapeutically effective agent in the presence of a polypeptide of the invention effective to disrupt cell-cell adhesion, cell-matrix adhesion, or both. Such co-administration can be in the form of a covalent complex, an ionic complex, a mixture simultaneous but separate administration, or administration within a relatively close temp sequence. Appropriate chemotherapeutic agents include, but are not limited to doxorubicin, daunorubicin, doxorubicin, idarubicin, vincristine, 6-mercaptopurine, 6-thioguanine, methotrexate, cytoxan, cytarabine, L-asparaginase, busulfan, cyclophoshamide, melphalan, carmustine, lomustine, 5-fluorouracil methotrexate, fludarabine, bleomycin, docetaxel etoposide, vinorelbine, antibodies, and the like.

Polypeptides of the invention can also be used in contraception methods. During pregnancy, the endometrial layer of the uterus becomes thickened and engorged with blood vessels upon implantation of a fertilized ovum. Without a well-developed vasculature the fertilized ovum will not be sustained, and the endometrial layer will be sloughed-off in the form of menses, i.e., menstruation. In one embodiment of the present invention, therapeutic compositions for use as contraceptives are provided. In order to induce contraception, the internal vasulature of the uterus is contacted with a contraceptively effective amount of a polypeptide of the invention. The mode of achieving bioavailability of polypeptides of the invention in this and other angioproliferative conditions can be, for example, through systemic or localized administration, such as intrauterine infusion.

Polypeptides of the invention can also be used to facilitate passage of compounds, such as pharmaceuticals, through the blood-brain barer of a mammal. A polypeptide of the invention can be administered along with a compound to permeablize a blood-brain barrier and allow delivery of the compound thought the blood-brain barrier. Such methods comprise co-administering a compound in the presence of a polypeptide of the invention effective to disrupt cell-cell adhesion, cell-matrix adhesion, or both. Such co-administration can be in the form of a covalent complex, an ionic complex, a mixture, simultaneous but separate administration, or administration within a relatively close temporal sequence.

Pharmaceutical Compositions and Administration Thereof

A pharmaceutically effective amount refers to an amount effective in treating an angiogenic condition in a mammalian patient, such as a human. A pharmaceutically acceptable excipient is a non-toxic carrier, vehicle or adjuvant that can be administered to a patient, together with a polypeptide of this invention, and which does not destroy the pharmacological activity of the polypeptide. Excipients, including carriers, vehicles and adjuvants are well known in the art See, e.g., Remington's Pharmaceutical Sciences, 17th Edition, Mack Publishing Co., Easton Pa., 1985. A pharmaceutically effective amount is an amount of a polypeptide that achieves a specified functional result. A dosage of a polypeptide of the invention can be determined using routine experimentation Pharmaceutical compositions of the invention can be administered by any number of routes including, but not limited to oral controlled release, intravenous, intramuscular, intra-arterial, intramedullary, intradermal intrathecal intraventricular, transdermal, subcutaneous, intraperitoneal, intranasal parenteral, topical, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, sips, slurries, suspensions, and the like, for ingestion by the patient.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol or dextran. Additionally, suspensions of the active compounds can be prepared as appropriate oily injection suspensions. Suitable lipophilic solvents or vehicles include fatty oils such as sesame oily or synthetic fatty acid esters, such as ethyl oleate or triglycerides, or liposomes. Non-lipid polycationic amino polymers also can be used for delivery. Optionally, the Z ion also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

A polypeptide of the invention can also be delivered to a patient by administration of a polynucleotide encoding a polypeptide of the invention. Preferably, injection of a P. gingivalis protease or hemagglutinin polynucleotide, which encodes a polypeptide of the invention, is used to treat, ameliorate, or prevent an angioproliferative condition. In addition to the practical advantages of simplicity of construction and modification, injection of a polynucleotide results in the synthesis of a polypeptide of the invention in the host. The polynucleotide is preferably delivered as “naked DNA” or in a vector. A vector can be a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma. Minichromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell). A polynucleotide can be, for example, injected intramuscularly to a mammal at a dose of 0.5, 0.75, 1.0, 1.5, 2.0, 2.5, 5 or 10 mg/kg.

In vivo delivery of polynucleotides (e.g., plasmid DNA) into results in the cellular uptake and expression of the polynucleotide into a desired polypeptide (Wolff, J. A. et al., Science 247:1465-1468 (1990); Wheeler, C. J. et al, Proc. Natl. Acad. Sci. USA 93:11454-11459 (1996)). The efficiency of in vivo polynucleotide administration can be increased using, for example, chemical agents or physical manipulations. Such chemical agents include cellular toxins such as bupivacaine, cardiotoxin or barium chloride, polymers such as polyvinyl pyrolidone, polyvinyl alcohol polyethyleneimine, polyamidomine, and polyethylene glycol-polyethyleneimine-transferrin complexes that coat the DNA and protect it from DNases and enhance plasmid DNA-based expression or immune responses, particles that interact with the DNA and act as carriers and enhance DNA expression such as nanospheres, microspheres, dendrimers, collagen and polylactide co-glycolides, bulking agents such as sucrose, detergents such as sodium glycocholate, sodium deoxycholate, and beta-cyclodextrin, cationic or non-cationic lipids, DNA binding agents, or agents that enhance plasmid DNA transcription such as histone deacetylase inhibitor FR901228 or S-Bromo-cyclic AMP. Physical manipulations include removal of nerves that control muscle contraction, electroporation, use of intravascular pressure, use of sutures coated with plasmid, use of sponges soaked with DNA as intramuscular depots to prolong DNA delivery, use of special needle-based injection methods, and of needleless-injectors tat propel the DNA into cells.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Because of the specificity of polypeptides of the invention for rapidly developing vasculature, it is possible to define doses of the polypeptides that are non-toxic to the remainder of a patient, but which nonetheless provide a localized effect to achieve beneficial anti-angiogenic effects. Dosages of a polypeptide of the invention can be about 0.01, 0.1, 1, 2, 5, or 10 mg/kg, of body weight, but one of ordinary skill in the art would recognize that for particular applications, it can be necessary to use either greater or lesser dosages. If a polypeptide of the invention is found to generate an immune response in a patient, processes are known in the art for mimicking biological activity of proteins through development of minienzymes, DNA agents, or other small molecules, repeat administration of active site mimics including for chronic administration.

The above disclosure generally describes the present invention, and all patents, patent applications, scientific references cited in this disclosure are expressly incorporated herein. A more complete understanding can be obtained by reference to the following specific examples which are provided for purposes of illusion only and are not intended to limit the scope of the invention.

### EXAMPLE 1

Methods for Testing Human Endothelial or Carcinoma Cell Detachment Exposed to P. gingivalis Extract

To determine the efficacy of P. gingivalis protease extracts in inhibiting cell proliferation, two cell lines were tested. Proliferation inhibition was assessed by determining the detachment of tissue culture cells from their substrate. A549 human non-small cell lung carcinoma cell line (maintained in RPMI-1640 medium (Gibco #11875-135) supplemented with L-Glu and Pen/Strep) and human endothelial cell line (HUVEC, ATCC #ECV-304), which was maintained in M199 medium, supplemented with L-Glutamine (0.05%, Gibco 25030-081), 0.1% Penicillin-Streptomycin (Gibco 15140-122) and 10% PBS (HyClone #SH30071.02, added after heat treatment at 56° C. for 30 min), were used as targets in a cell detachment assay. Exponentially growing (Ex) and quiescent (Plateau) phases of cell culture growth were tested. To obtain extract for activity tests, exponentially grown broth cultures of P. gingivalis strain W83 were pelleted, resuspended in 50 mM HEPES buffer (pH 7.5), and sonicated on ice for 2 min. Following centrifugation at 14,000 rpm at 4 C, the supernatant was filtered through 0.22 μm non-protein-binding filter (Gelman) and stored at −80° C. until needed. Protein concentration was measured using Sigma bicinchoninic acid reagent (#B-9643), BSA standard solution and spectrophotometer Shimadzu UV-1201.

Six plates per sample with three plates added for non-treated control were used. 10⁴ cells per 60-mm tissue culture plate were used to seed the sample. Detached cells were counted by collecting the medium after treatment (24 or 48 hrs), washing the attached cells with calcium-free phosphate-buffered saline (PBS) and adding the wash to the medium. The pellet was then resuspended in medium to obtain a countable number on the hemocytometer. The 25-square (0.1 μl volume) count ×10⁴ gives the cell count per ml. Two readings were made of each of the three plates. This number represented the detached cell count.

The attached cells were trypsinized and collected as above; the plate was washed with PBS and added to the tube. After pelleting and resuspending, two readings were made per each of the three plates to obtain the attached cell count. The mean value of the counts was taken, total number of cells (attached+detached) was obtained and the percentage of detached cells was calculated. Different concentrations of P. gingivalis protein extract were used in the beginning to establish effective concentration to be used throughout. The working concentration was chosen to be 0.4 mg total protein tact per ml cute medium.

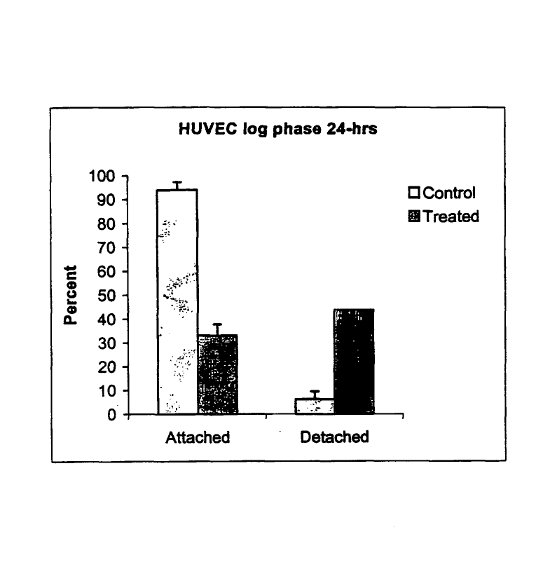

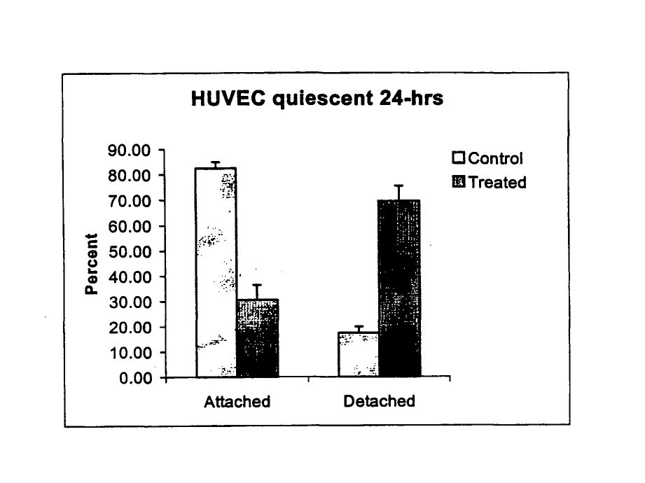

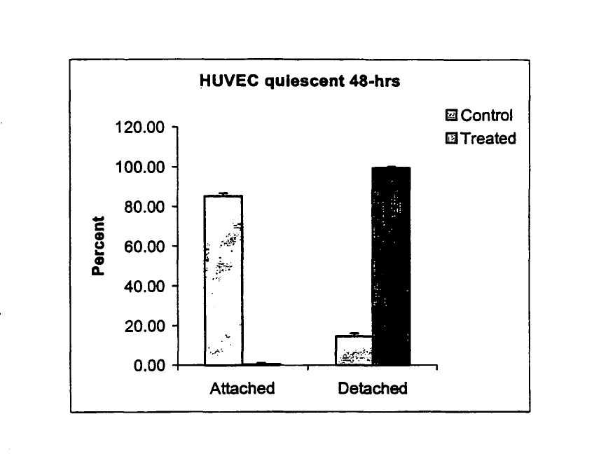

FIG. 1 and FIG. 2 show non-confluent (“log”) culture treated with 0.4 mg P. gingivalis extract/ml for 24 hours (FIG. 1) and 48 hours (FIG. 2). FIG. 3 and FIG. 4 show confluent culture treated for 24 hours (FIG. 3) and 48 hours (FIG. 4), respectively. FIG. 1 shows a 70% detachment for active HUVEC culture cells following treatment with a P. gingivalis extract over a 24-hour period. FIG. 2 shows a 90% detachment of the same cells after 48 hours. FIG. 3 shows approximately 68% detachment of quiescent Human (HUVEC after 24 hours of treatment, whereas the same cells exhibit nearly 100% detachment after 48 hours (see FIG. 4).

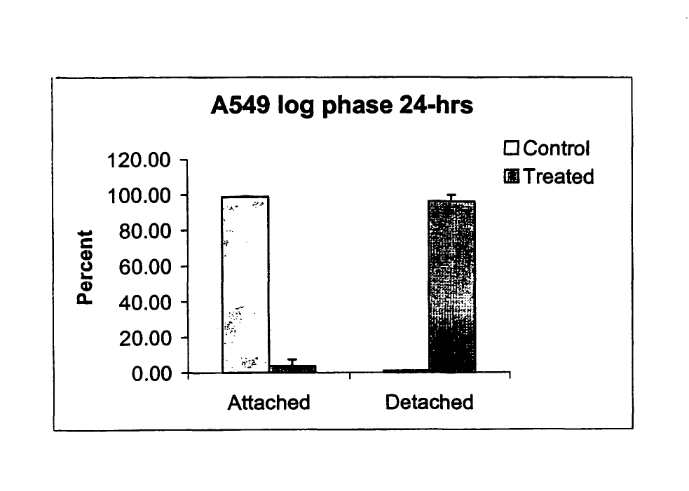

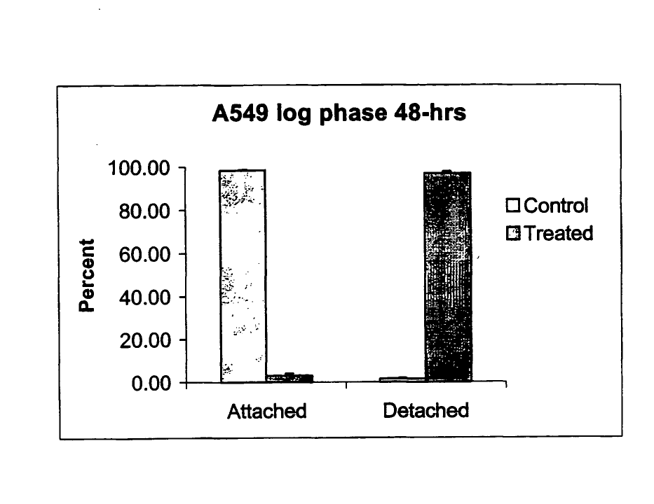

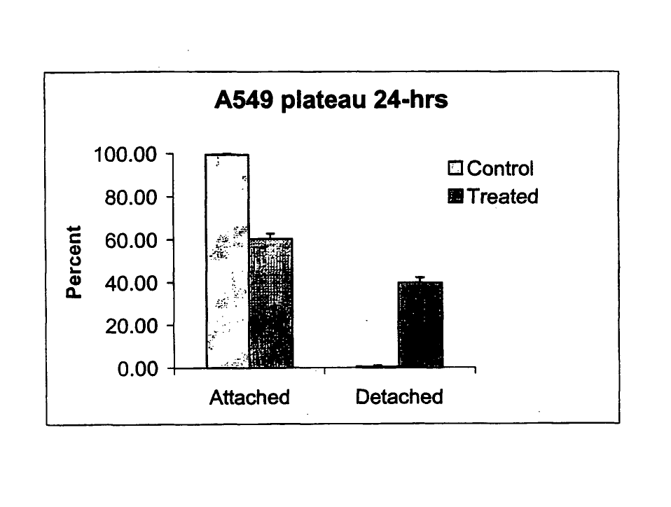

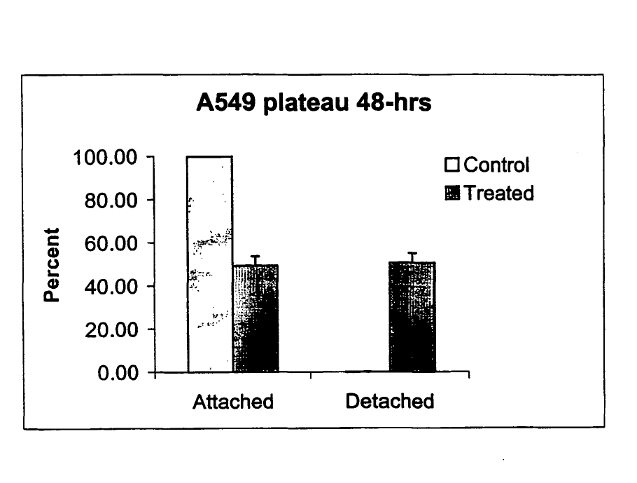

FIG. 5 and FIG. 6 show non-confluent (“log”) culture treated for 24 hours (FIG. 5) and 48 hours (FIG. 6) with 0.4 mg P. gingivalis extract/ml medium. FIG. 7 and FIG. 8 show confluent cultures treated for 24 hours (FIG. 7) and 48 hours (FIG. 8). The control is untreated A549. FIG. 5 shows active A549 human non-small cell lung carcinoma exhibited a 95% detachment rate after 24 hours of treatment with a P. gingivalis extract. After 48 hours, there was no change in the percent detachment of these same cells (see FIG. 6). FIG. 7 shows 40% detachment rate after 24 hours, whereas after 48 hours the detachment rose to greater than 50%. Detachment from the plastic surface of the petri dish is consistent with the ability of P. gingivalis extract to degrade β1 integrin. Anti-angiogenic and tumor-reducing activity correlates with the degree of detachment of the endothelial and cancer tissue culture cells in both 24 and 48-hour treatments. Demonstrating detachment of up to 96% of the cells (A549) or 9% (HUVEC) can thus be considered pertinent activity. This is for total protein where the fraction of the active ingredient is small. Therefore, its activity is very high. Both exponentially growing (“log”) and stationary phase (“plateau”) quiescent cultures showed differences in ability to remain attached following treatment

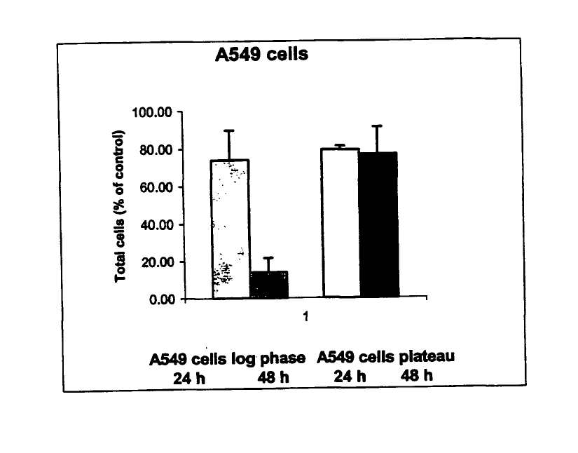

FIG. 15 total cell number reduction a result of the treatment. The treatment is more efficient on growing tumor cells than on cells in stationary phase. Similar reduction was obtained with endothelial (HUVEC) cells.

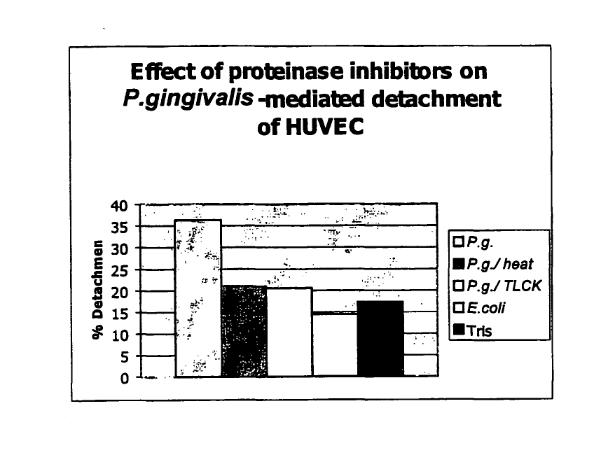

FIG. 16 demonstrates that detachment of HUVEC is reduced by inhibitors or heating of SPF (soluble protein faction; see Example 9). In order to determine if the junctional protein-degrading activity is a cysteine proteinase, additional treatments were included Nα-p-tosyl-L-lysine chloromethyl ketone (TLCK), a cysteine proteinase inhibitor, was added at 10 mM. In addition, heat-inactivation (20 min at 65° C.) of the P. gingivalis extract was performed to inactivate protein-mediated proteolysis. All experiments were done in triplicate. Using a Coulter counter, the number of the detached cells was determined and mean values were expressed as percent of the total (detached+attached) cell number. The results of this experiment demonstrate that a P. gingivalis protein is the active substance and that this protein is a cysteine proteinase.

### EXAMPLE 2

Method of Demonstrating Detachment Associated with PrtP Protease from P. gingivalis

To demonstrate that protease PrtP isolated from P. gingivalis is responsible for the detachment observed in Example 1, three samples were applied to A549 lung carcinoma cells. A single P. gingivalis protein, PrtP protease, was expressed in Bacteroides fragilis, a species related to P. gingivalis, but which does not express PrtP, for the purpose of further chromatographic purification. Treatment of carcinoma cells was performed with an extract of B. fragilis containing PrtP and compared to the same treatment with the wild-type B. fragilis host. Therefore, the difference between the treatments was limited to the presence/absence of P. gingivalis PrtP protease only.

The strains were grown in BHIS broth (per liter, 37 g Brain Heart Infusion (Difco), 1 g L-Cysteine (Sigma), 5×10⁻⁴% hemin, 0.2% NaHCO₃ in an anaerobic chamber with an atmosphere of 5% CO₂, 10% H2, and 85% N₂). Agar (1.5%) was added for solid medium. P. gingivalis W83 was grown on Trypticase soy agar (BBL Microbiology stems, Cockeysville, Md.) supplemented with sheep blood (5%), hemin (5 mg/ml), and menadione (5 mg/ml). When broth-grown P. gingivalis was required, cultures were grown in Todd-Hewitt broth (BBL Microbiology Systems) supplemented with hemin (5 μg/ml), menadione (5 μg/ml), and glucose 2 mg/ml) anaerobically. Normal Bacteroides fragilis was used as a control.

Detachment of cells exposed to both B. fragilis with PrtP and P. gingivalis were nearly equal, whereas those cells exposed to the control exhibited minimal detachment. This study provides direct evidence that PrtP is active in cell proliferation inhibition.

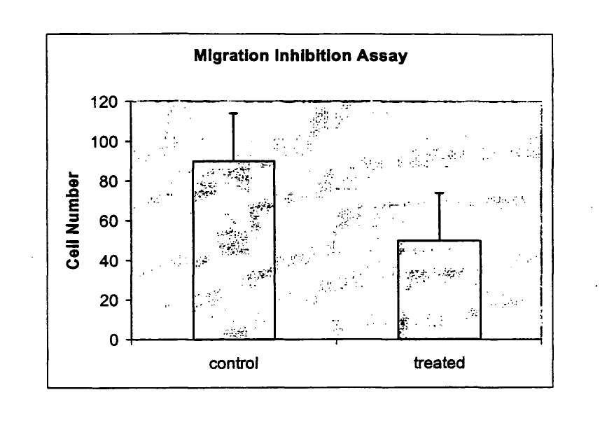

A migration inhibition (“scratch wound”) assay is a method that is routinely used for the ration of the ability of cells to migrate, an important step in tumor neovascularization. This assay is used by the Biological Testing Branch, Developmental Therapeutics Program at NCI to identify new anti-angiogenic compounds. Inhibition of migration is considered one aspect of anti-angiogenic activity (Yeh et al., Mol Pharmacol. 2001 May,59(5):1333-42.). FIG. 9 demonstrates a HUVEC migration inhibition assay: For this assay, endothelial cells were cultured on a slide. Upon reaching confluency, a 2-mm scrape “wound” was introduced on a central portion of the slide. The number of cells which migrated into the denuded area was enumerated after further incubation for 24 hours. The results (the mean of 2 experiments) demonstrate that at 0.4 mg total protein/ml, the migration of the human vascular endothelial cells was reduced by 45%.

### EXAMPLE 3

Migration Inhibition Assay

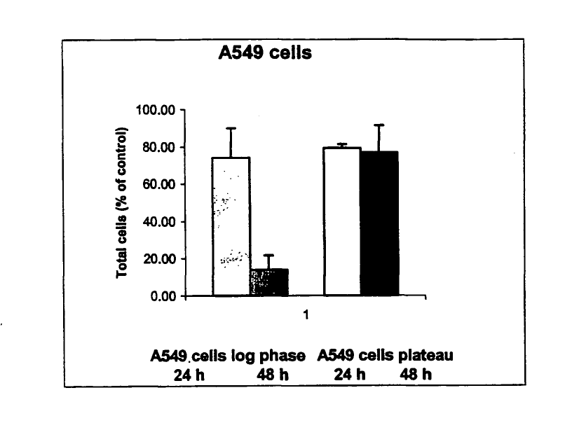

To demonstrate that a P. gingivalis extract exerts anti-angiogenic effects, as opposed to general inhibition of cell proliferation, the following assay was performed. P. gingivalis extracts were produced and homogenized to obtain an extract as described in Example 1. At 0.4 mg total protein/ml, human vascular endothelial cell migration in standard in vitro assay known in the at to reflect angiostatic and anti-tumor acidity, was reduced by 45%, (mean value of 2 experiments). In addition, at 48 hours, detachment of 85% of log phase lung carcinoma cells was observed (FIG. 10).

Since total cell protein was used, where the faction of the active ingredient is small, this experiment demonstrates that the angiostatic activity of the P. gingivalis proteinase is high.

### EXAMPLE 4

Identification of Epithelial Cell Ligands of Hemagglutinin A

In order to determine if HagA interacts directly with host cell components, a functionally active fragment of HagA was produced in E. coli using the E. coli expression vector, pET19b (Novagen). In this system, purification was achieved by fusing a histidine tag to a Hag fragment and by affinity purification of the fusion protein on a Ni²⁺ column. Oligonucleotides were designed flanking 2 HArep sequences to include the active site of hemagglutination as disclosed in U.S. Pat. No. 5,824,791 and to include restriction sites for ligation of the fragment into the expression vector, pET19b. The 3 kb PCR product was cloned into pT7Blue vector (Novagen), digested with NdeI and XhoL and the coding sequence directionally subcloned into pET19b, which had been digested with the same enzymes and CIP-treated.

Using PCR with a mixed pair of primers T7 (from vector) and ST2/3′ (from insert), transformants in E. coli Novablue (Novagen) were screened for an insert in the proper orientation. One such clone, pEKS5, was chosen for further work and was transformed into E. coli BL21 (DE3), an expression strain (Novagen). After induction with 1 mM IPTG, cells were lysed and the lysate was applied to an activated His-Bind resin affinity chromatography column. Elution with 1 M imidazole-containing buffer produced a single protein species with an apparent molecular mass of ˜100 kDa. After transfer onto a nitrocellulose membrane, the protein was probed with anti-HagA antibody, 61BG1.3, and its authenticity was confirmed.

The purified recombinant HagA peptide was tested for binding to cell components of two human cell lines using the Far Western Immunoblot. For this assay, KB oral epithelial cells and human umbilical cord endothelial cells (HUVEC) were grown and lysed in hypotonic buffer containing a cocktail of mammalian proteinase inhibitors. The cell lysates were loaded on SDS-PAGE gels, transferred to nitrocellulose membranes, blocked with dry fat-free milk in TBS, and overlaid with 0.5 μg/ml of purified recombinant HagA. After three hours of incubation at ambient temperature followed by washing, the membranes were treated first with anti-HagA Mab and secondly with anti-mouse AP conjugate. The HagA peptide was found to bind intensely to two proteins, ˜60 kDa and 65 kDa in size, present in both epithelial and endothelial cells. The HagA peptide also bound to two heavy protein species, >200 kDa, present in endothelial cells. These results demonstrate that HagA binds to and int with one or more proteins present in host cells and suggest the in vivo existence of a protein complex between HagA and endothelial as well as epithelial proteins.

### EXAMPLE 5

Proliferation Inhibition of HUH7 Cells by P. gingivalis Extracts, and by Live P. gingivalis Cells in the Presence/Absence of Inhibitors

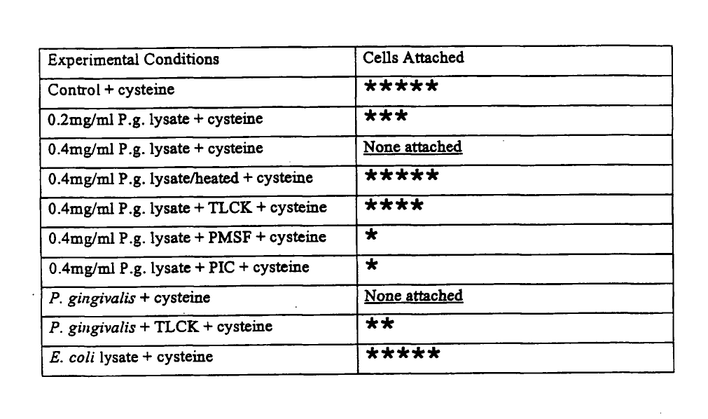

Freshly collected whole P. gingivalis cells were used for 20-hour treatment at a density of 2×10¹⁰ bacteria per ml DMEM (antibiotic-free). FIG. 11 represents the proliferation inhibition of HUH7 cells by P. gingivalis and E. coli extracts, and by live P. gingivalis cells in the presence/absence of inhibitors (five stars: all cells remain attached, no proliferation inhibition). L-Cysteine was always present at concentration of 5 mM to stabilize the anti-angiogenic activity. The proliferation inhibition property of P. gingivalis extract and whole cells is clearly demonstrated on human hepatoma cell line (HUH7).

### EXAMPLE 6

Immunofluorescent Microscopy of HUH7 Human Hepatoma Cells Treated with P. gingivalis Extract

Tissue culture cells grown in T-75 flasks at 37 C in DMEM (Pen/Strep) in a CO₂ incubator were subjected to 8 ml of trypsin-EDTA and incubated at 37 C for 10-15 minutes for detachment. Trypsinized cells were transferred (with 2×10 ml DMEM) to 50 ml culture tube and centrifuged at 1K rpm for 10 seconds. Supernatant was removed and cells were washed at 8 ml Ca-free PBS. Washed cells were centrifuged at K rpm for 10 seconds and excess wash was removed. Cells were resuspended in 20 ml of DMEM (Pen/Strep) media and transferred to new T-75 flask for incubation at 37 C in 5% CO. HUH7 cells were incubated with P. gingivalis extract (0.8 mg protein per ml of medium) for 20 h at which time the cells were washed three times with phosphate buffered saline (PBS) and then fixed in 4% paraformaldehyde in PBS for 30 minutes at room temperature. This was followed by washing twice in PBS and quenching in NH₄Cl (50 mM)/0.3% Tween 20/PBS for 10 minutes at room temperature. After quenching, the HUH7 were washed two times in PBS. The primary antibodies were rabbit anti-human occluding (Zymed Laboratories #71-1500) and rabbit anti-human pan cadherin (Sigma Chemical Co., St Louis, Mo. #C3678). They were diluted 1/50 in PBS/5% normal goat serum/0.3% Tween™ 20 and applied to the cells for 2 h at room temperature. The HUH7 were then washed four times in PBS for 5 minutes each time. The secondary antibody (rhodamine-conjugated goat anti-rabbit (Sigma)) was applied for 1 h at room temperature. The HUH7 cells were then washed twice with PBS before mounting with Fluoromount-G (Southern Biotechnology Associates, Inc., Birmingham Ala.) onto glass microscope slides and sealing with nail polish. Images were viewed using an Olympus IX70 deconvolution microscope and Delatvision software (Applied Precision, Inc., Wepahah, Wash.).

### EXAMPLE 7

Reactivity with Anti-Occludin Antibody

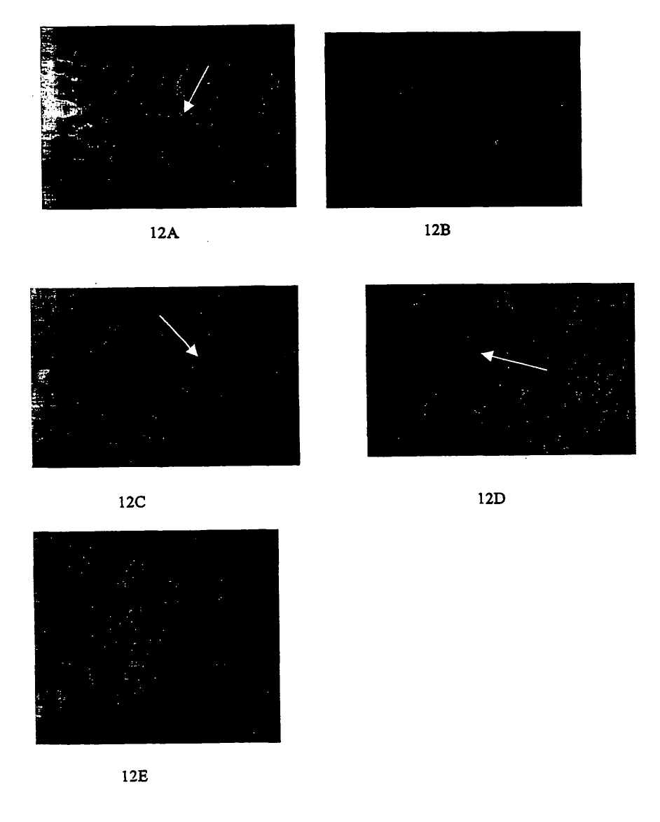

Occludin-stained junctions degraded upon treatment with P. gingivalis extract. TLCK presence or heat-inactivation of P. gingivalis extract abolishes the activity. E. coli extract control treatment exhibited no activity and the occludin network was intact. FIG. 12A shows non-treated HUH7 cells. FIG. 12B shows HUH7 cells after treatment with P. gingivalis extract wherein the occludin network was degraded; FIG. 12C shows HUH7 cells after treatment with P. gingivalis extract in the presence of inhibitor TLCK, wherein the occludin network was intact; FIG. 12D shows HUH7 cells following treatment with heat-treated P. gingivalis extract, wherein the occludin network was degraded; and FIG. 12E shows HUH7 cells treated with E. coli extract and demonstrates that E. coli does not effect the occludin network. The data from immunoflourescent staining or junctional molecules from HUH7 cells confirms the capacity of P. gingivalis extract to disrupt the intracellular network by degrading the border consisting of cell adhesion molecules (CAMs).

### EXAMPLE 8

Reactive with Anti-Pan Cadherin Antibody

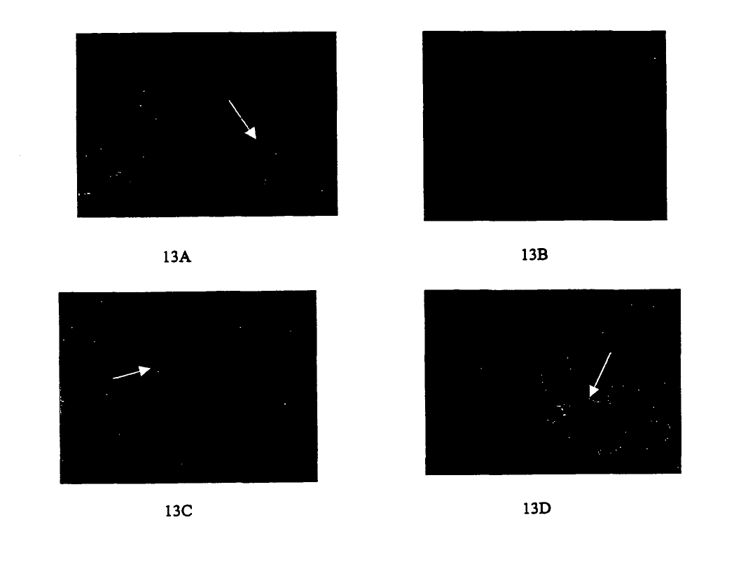

Cadherin-stained junctions were degraded upon treatment with P. gingivalis extract. TLCK presence or heat-inactivation of P. gingivalis abolish the activity. FIG. 13A shows control, non-treated HUH7 hepatoma cells, wherein the junctions were intact FIG. 13B shows HUH7 cells following treatment with P. gingivalis extract, wherein the junctions were degraded; FIG. 13C shows HUH7 cells after treatment with P. gingivalis extract in the presence of inhibitor TLCK, wherein the junctions were intact; and FIG. 13D shows HUH7 cells after treatment with heat-inactivated P. gingivalis extract, wherein the junction were intact. The proliferation as stated above, and the data from immunoflourescent staining of junctional molecules from HUH7 cells confirms the capacity of P. gingivalis extract to disrupt the intracellular network by degrading the border consisting of cell adhesion molecules. Thus both examples 7 and 8 demodulate that the unique activity of the present disclosed extract can be utilized for tumor disintegration.

### EXAMPLE 9

Fractioning of Bacterial Culture Liquor Proteins

In order to partially purify the CAM-degrading activity, fractional precipitation of secreted P. gingivalis proteins from spent culture liquor was achieved using ammonium sulfate. Broth culture grown in an anaerobic chamber was centrifuged for 20 minutes at a speed of 8000 rpm. Next it was filtered with a 0.2 μm filter (Nalgene) to remove any remaining cells. To saturate to 60%, 36.1 g of (NH₄)₂SO₄ were dissolved in every 100 ml of culture liquor. The solution was left stirring overnight at 4 C and collected the next day by centrifuging for 20 minutes at 8000 rpm. Precipitated proteins were collected from six liters of P. gingivalis W83 spent culture medium. The protein pellet was resuspended in a 20 ml solution of 50 mM Tris HCl (pH 7.5). The solution was dialyzed (Pierce SnakeSkin tubing, 7 kDa MWCO) against 50 mM Tris.HCl overnight at 4 C The dialysis was repeated with fresh buffer. Dialyzed solution was filtered with an Acrodisc® syringe filter (0.2 μm) and then concentrated using a Centriprep 10 (Amicon) for a total of an hour and a half After concentration, the solution was aliquoted, and the protein concentration was determined using the BCA assay (Sigma B-9643). The solution was then stored at −80 C

### EXAMPLE 10

Proliferation Inhibition of HUVEC Polarized Cell Line. HUVEC #Ecv-304 Treatment with P. gingivalis Fractions

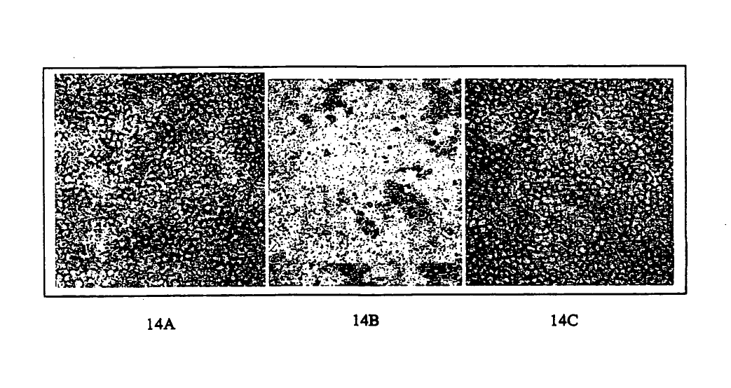

Polarized endothelial cells cultured on porous membrane inserts (Transwell, Corning Costar Corp., Cambridge, Mass.) were used as an in vitro model for studying anti-angiogenic activities and to test for differential activity from both sides of the endothelium. Six hundred id (for 24-well plate) or 2.6 ml (for 6-well plate) of DMEM medium (Penn/Strep) were added to the lower chamber of tissue culture plates. Vascular endothelial cells were seeded into Corning Costar Transwell inserts in volumes of 0.1 ml medium (24-well plate) or 1.5 ml medium (6-well plate) in the upper chamber. Cultures were grown to confluence in a CO₂ incubator at 37.0 C before being treat The proteins were added to the upper or lower chambers at a final concentration of 0.8 mg/ml. The cultures were incubated for 4 days in a CO₂ incubator. Similar rets were obtained using whole P. gingivalis cells (data not shown). FIG. 14A shows control, untreated polarized human endothelial cells ECV-304; FIG. 14B shows polarized ECV-304 cells treated basolaterally with 60% fraction of P. gingivalis culture liquid proteins; and FIG. 14C shows polarized ECV-304 cells following treatment lumenally with 60% fraction of P. gingivalis culture liquid proteins.

In each experiment the polarized endothelial cell layer was treated from either the apical or basolateral side with identical concentrations of protein preparations. As seen in the optical micrographs in FIGS. 14A, B and C at a point where complete destruction was observed from basolateral application of ammonium sulfate-precipitated proteins, no damage was observed in the cultures with lumenal (apical) application of same preparations. These data strongly support the conclusions that anti-angiogenic activity is partially purified from P. gingivalis secreted proteins as 60-% fraction of ammonium sulfate-precipitated culture liquor proteins; and the targeting of this activity toward the basolateral, extravascular side of the vasculature is specifically beneficial for degradation of the endothelial vascular cell layer in abnormally leaky tumor vessels. In addition to anti-angiogenic activity, immunofluorescent and proliferation inhibition studies with human cancer cell lines (hepatoma and lung carcinoma) demonstrate the utility of this P. gingivalis-associated activity for disintegration of extravascular tumor tissues, i.e., direct tumor-disintegration activity exists. Using the same abnormal openings to access both the basolateral side of the tumor vasculature and the surrounding tumor tissue brings double benefit to the proposed treatment.

In light of foregoing evidence, it is apparent that P. gingivalis polypeptides of the invention can be utilized as a vascular endothelial cell migration inhibitor and as an anti-angiogenic pharmaceutical agent Furthermore, while at present there does not appear to be any known therapeutic protocol based on selective degradation of cell-cell and cell-matrix adhesion molecules in tumors and a large number of other diseases, the present invention provides a new method of disease treatment of such pathologies. It is further predictable, based on the disclosure provided herein, that other Porphyromonas gingivalis arginine or lysine specific cysteine protease polypeptides, hemagglutinin polypeptides and fragments thereof known or yet to be discovered that exhibit similar anti-angiogenic activity, can be used according to the methods of this invention. Furthermore, combinations of such molecules can also be used according to the methods of this invention

### EXAMPLE 11

Effect of P. gingivalis on Integrin Receptors.

α5β1 integrin is upregulated in tumor endothelium (Stupack and Cheresh, Sci STKE. 2002 Feb. 12;2002(119):PE7). Being an apoptosis regulator, the integrin is a target for anticancer drugs. B1 integrin is a target of P. gingivalis proteolytic activity in canine epithelial cells (Katz, et al., Infect Immun. 2000 Mar. 68(3):1441-9). To test if β1 integrin is targeted in a human cell line, immunoanalysis of detached HUVEC was performed after treatment with the P. gingivalis extract to determine the extent of degradation. Western blots of SDS-PAGE gels were probed using anti-human β1 integrin MAb (BD Transduction Labs, cat. # MMS-496R). The results demonstrated that treating with the P. gingivalis extract (except if heat-inactivated) resulted in complete degradation of the 130-kDa integrin.

### EXAMPLE 12

Effect of P. gingivalis Proteins on Transendothelial Resistance (TER) of Polarized HUVE Cells

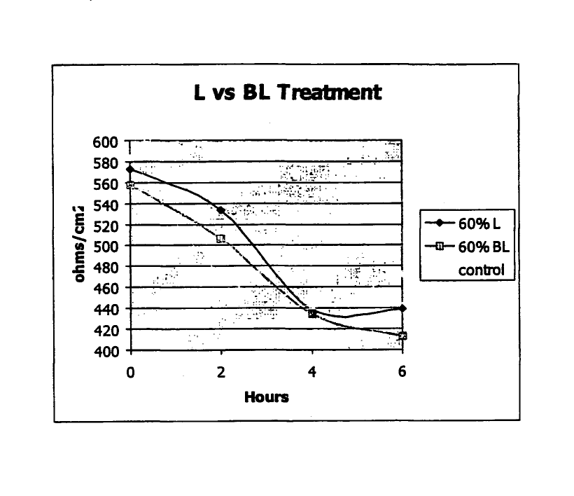

Junctional complexes between adjacent polarized endothelial cells constitute a permeability barrier between lumenal and basolateral compartments. Disruption of the junctional complexes leads to increased flow of solutes, including small ions that can be monitored electrophysiologically by reading the changes in the transendothelial resistance (TER). HUVE cells were grown onto Transwell inserts with 0.4 μm pore size. The integrity of the HUVEC layer was decreased by treatment with P. gingivalis SPF compared to mock-treated control See FIG. 17 (the data are mean values from triplicate experiments). Notably, an identical concentration of SPF applied to the BL (basolateral) compartment decreased TER faster than when applied to the L (lumenal) compartment thus confirming and extending the observations made with canine epithelial cells. (Katz et al., Infect Immun. 2000 March 68(3):1441-9.)

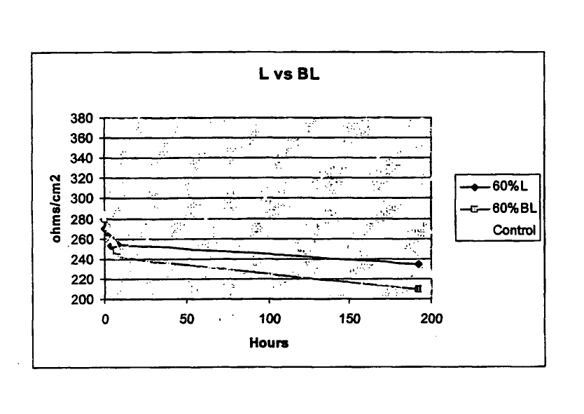

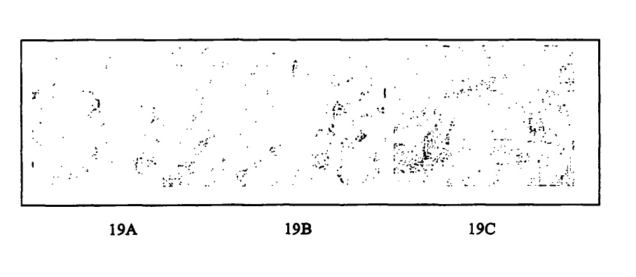

FIG. 18 shows TER data and micrographs for an extended period of time (8 days). Similar HUVEC treatment as above was performed in triplicate. FIG. 19 shows the TER graph FIG. 19 shows contrast micrographs of the cell layers on day 8. FIG. 19A; L-treated; FIG. 19B, BL-treated and FIG. 19C, mock-treated control. The difference in the TER level reflects the status of the endothelial layer as documented on the micrographs, totally non-existing (BL treatment) and virtually intact (L treatment; control). Serine proteinase (trypsin) treatment (0.05% in medium) does not provide such specificity. Using electrophysiology and microscopy on the same cell cultures, these data again demonstrate the destructive action of P. gingivalis secreted proteins on polarized human endothelial cell model and their preference for the BL side of the cell layer.

### EXAMPLE 13

Endothelial Degradation Following Treatment with 15-kDa P. gingivalis Protein Cloned in E. coli.

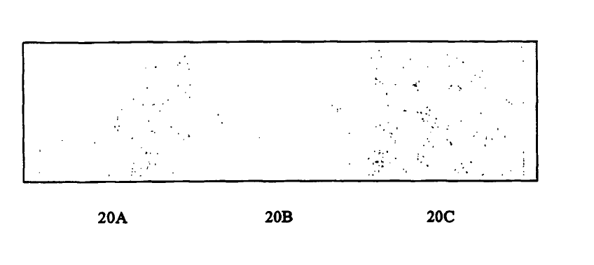

A 15-kDa internal fragment of a HagA repeat (described in Paramaesvaran, et al., 1998, J. Dental Res., 77:664) was expressed in E. coli. In this experiment, a confluent endothelial cell layer was treated with a soluble sonic extract from E. coli expressing 15-kDa subunit of the adhesive domain (of gingipains or HagA), 0.5 mg total protein/mL FIG. 20 shows micrographs of endothelial cell layer. FIG. 20A shows HUVEC treated with control E. coli host extract, 0.5 mg protein/mL FIG. 20C shows control mock-treated cells. FIG. 20B shows eminent with E. coli expressing 15-kDa protein.

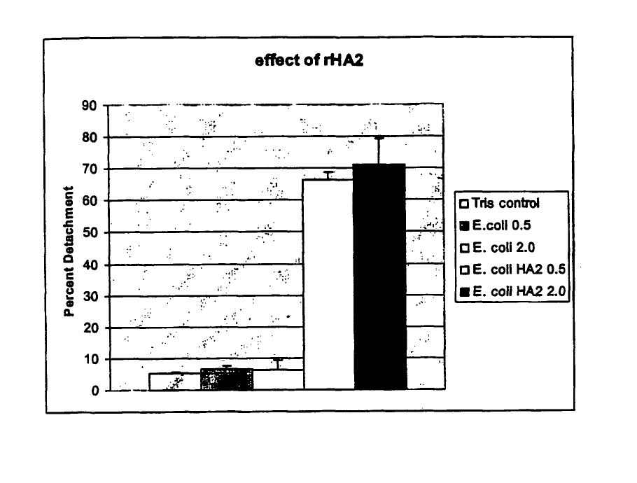

FIG. 21 shows proliferation inhibition of HUVEC treated with 15-kDa P. gingivalis protein cloned in E. coli. (E. coli 0.5, control E. coli protein extract at 0.5 mg/ml. E. coli HA2 0.5, E. coli protein extract containing HA2, the 15-kDa polypeptide at 0.5 me). The results of this experiment demonstrate sharp reduction of cell numbers upon treatment with recombinant 15-kDa P. gingivalis protein.

### EXAMPLE 14

P. gingivalis cysteine proteinases and hemagglutinins can mediate detachment of cells from a substrate and inhibit proliferation in human endothelial and carcinoma cells. Also, junctional molecule degradation and vascular network disintegration using bacterial extracts and whole cells has been demonstrated. Further, partially purified secreted junctional molecules-targeting proteinase have specificity toward the basolateral side of polarized human endothelial monolayer.

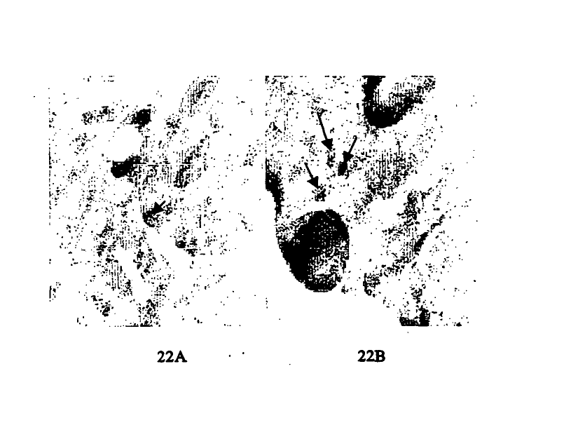

In addition to the endothelial cell experiments, experiments with human cancer cell lines demonstrate the utility of this P. gingivalis-associated activity for treatment of extravascular tumor tissues, i.e. direct tumor disintegration in addition to anti-angiogenic activity. This activity can be specifically beneficial for degradation of the endothelial vascular cell layer from the basolateral side in the abnormally leaky tumor vessels. See FIG. 22. Using same abnormal openings to access both the basolateral side of the tumor vasculature and the extravascular tumor tissue would bring double benefit to the proposed treatment.

FIG. 22 shows potential pathways for leakage from blood vessels in tumors. Intercellular openings (arrows) between lining cells of a murine tumor vessel viewed by scanning electron microscopy. Figures A and B show multiple large intercellular openings (arrow, 22A) and three smaller transcellular holes (arrows, 22B) in branched lining cells of a tumor vessel. The intercellular openings are much larger than the holes. Region in box in 22A is shown at higher magnification in 22B. (Hashizume, et al., Am J Pathol. 2000 Apr. 156(4):1363-80.)

1. A composition for the treatment, prevention, or amelioration of an angioproliferitive condition comprising a pharmaceutically effective amount of a substantially purified Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof, wherein the polypeptide has anti-angiogenic activity, and a pharmaceutically acceptable excipient.

2. The composition of claim. 1, wherein the polypeptide is selected from the group consisting of rgpA, rgpB, kgp, hag, prtT and tla polypeptides.

3. The composition of claim 2, wherein the polypeptide has at least about 90% sequence identity with a rgpA, rgpB, kgp, hag, prtT or tla polypeptide and wherein the polypeptide has anti-angiogenic activity.

4. The composition of claim 2, wherein the polypeptide is a biologically functional homolog, or isoform of a rgpA, rgpB, kgp, hag, prtT or tla polypeptide.

5. The composition of claim 1, wherein the polypeptide is a fragment of a Porphyromonas gingivalis arginine specific cysteine protease polypeptide, Porphyromonas gingivalis lysine specific cysteine protease polypeptide, or Porphyromonas gingivalis hemagglutinin polypeptide, and wherein the fragment has anti-angiogenic activity.

6. The composition of claim 5, wherein the fragment is HA2.

7. The composition of claim 1, wherein the angioproliferative condition is carcinoma, sarcoma, melanoma, benign tumor, ocular retinopathy, retrolental fibroplasias, psoriasis, angiofibromas, endometriosis, hemangioma, rheumatoid arthris, Osler Webber Syndrome, myocardial angiogenesis, telangiectasia, hemophiliac joints, wound granulation, intestinal adhesions, post-surgery adhesions, arteriosclerosis, scleroderma, hypertrophic scars, cat scratch disease, Helicobacter pylori ulcers, capillary proliferation within atherosclerotic plaque, or a combination thereof.

8. A method for the treatment, prevention, or amelioration of an angioproliferative condition comprising administering a pharmaceutically effective amount of a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof wherein the polypeptide has anti-angiogenic activity, to a patient in need thereof whereby the angioproliferative condition is treated, prevented or ameliorated.

9. The method of claim 8, wherein the polypeptide is selected from the group co-ng of rgpA, rgpB, kgp, hag, prtT and tla polypeptides.

10. The method of claim 8, wherein the polypeptide has at least about 90% sequence identity with a rgpA, rgpB, kgp, hag, or tla polypeptide and wherein the polypeptide has anti-angiogenic activity.

11. The method of claim 8, wherein the polypeptide is a fragment of a Porphyromonas gingivalis arginine specific cysteine protease polypeptide, Porphyromonas gingivalis lysine specific cysteine protease polypeptide, or a Porphyromonas gingivalis hemagglutinin polypeptide, ad wherein the fragment has anti-angiogenic activity.

12. The method of claim 11, wherein the polypeptide fragment is HA2.

13. The method of claim 8, wherein the angioproliferative condition is carcinoma, sarcoma, melanoma, benign tumor, ocular retinopathy, retrolental fibroplasias, psoriasis, angiofibromas, endometriosis, hemangioma, rheumatoid arthritis, Osler Webber Syndrome, myocardial angiogenesis, telangiectasia, hemophiliac joints, wound granulation, intestinal adhesions, post-surgery adhesions, atherosclerosis, scleroderma, hypertrophic scars, cat scratch disease, Helicobacter pylori ulcers, capillary proliferation within atherosclerotic plaque, or a combination thereof.

14. The method of claim 8, wherein the method comprises contacting a vasculature supplying a biological structure affected by the angioproliferative condition with the polypeptide.

15. The method of claim 14, wherein the polypeptide is contacted with a basolateral surface of the vasculature.

16. A method for potentiating effects of a chemotherapeutically effective agent comprising administering to a patient:
(a) a Porphyromonas gingivalis arginine specific cysteine protease polypeptide, Porphyromonas gingivalis lysine specific cysteine protease polypeptid, Porphyromonas gingivalis hemagglutinin polypeptide, or a combination thereof, wherein the polypeptide disrupts cell-cell adhesion, cell-matrix adhesion, or both; and
(b) a chemotherapeutically effective agent,
whereby effects of a chemotherapeutically effective agent are potentiated.

17. The method of claim 16, wherein the polypeptide is selected from the group consisting of rgpA, rgpB, kgp, hag, prtT and tla polypeptides.

18. The method of claim 17, wherein the polypeptide has at least about 900% sequence identity with a rgpA, rgpB, kgp, hag, or tla polypeptide, and wherein the polypeptide has anti-angiogenic activity.

19. The method of claim 16, wherein the polypeptide is a fragment of Porphyromonas gingivalis arginine specific cystine protease polypeptide, Porphyromonas gingivalis lysine specific cysteine protease polypeptide, or Porphyromonas gingivalis hemagglutinin polypeptide, and wherein the fragment has proteinase activity.

20. The composition of claim 19, wherein the polypeptide fragment is HA2.

21. A method for preventing the formation of new vasculature required for implantation or sustenance of a fertilized an ovum comprising:
administering a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof to a mammal, wherein the polypeptide has anti-angiogenic activity, whereby the formation of new vasculature required for implantation or sustenance of a fertilized mammalian ovum is prevented.

22. The method of claim 21, wherein the polypeptide is selected from the group consisting of rgpA, rgpB, kgp, hag, prtT and tla polypeptides.

23. The method of claim 22, wherein the polypeptide has at least about 90% sequence identity with a rgpA, rgpB, kgp, hag, prtT, or ta polypeptide and wherein the polypeptide has anti-angiogenic activity.

24. The method of claim 21, wherein the polypeptide is a fragment of a Porphyromonas gingivalis arginine specific cysteine protease polypeptide, Porphyromonas gingivalis lysine specific cysteine protease polypeptide, or Porphyromonas gingivalis hemagglutinin polypeptide, and wherein the fragment has anti-angiogenic activity.

25. The method of claim 24, wherein the polypeptide fragment is HA2.

26. A pharmaceutical composition for facilitating passage of a compound through a blood-brain barrier comprising a pharmaceutically effective amount of a substantially purified Porphyromonas gingivalis arginine specific cysteine protease polypeptide, Porphyromonas gingivalis lysine specific cysteine protease polypeptide, a Porphyromonas gingivalis hemagglutinin polypeptide, or a combination thereof, and a pharmaceutically acceptable excipient.

27. The pharmaceutical composition of claim 26 further including a compound to be passed through the blood-brain barrier.

28. A method delivering a compound through a blood-brain barrier of a patient comprising administering a pharmaceutically effective amount of a Porphymonas gingivalis arginine specific cysteine protease polypeptide, Porphyromonas gingivalis lysine specific cysteine protease polypeptide, Porphyromonas gingivalis hemagglutinin polypeptide, or a combination thereof and the compound, whereby the compound is passed through the blood-brain barrier.

29. The method of claim 28, wherein the polypeptide is selected from the group consisting of rgpA, rgpB, kgp, hag, prtT and tla polypeptides.

30. The method of claim 29, wherein the polypeptide has at least about 90% sequence identity with a rgpA, rgpB, kgp, hag, prtT or tla polypeptide and wherein the polypeptide has anti-angiogenic activity.

31. The method of claim 28, wherein the polypeptide is a foment of a Porphyromonas gingivalis arginine specific cysteine protease polypeptide, Porphyromonas gingivalis lysine specific cysteine protease polypeptide, or a Porphyromonas gingivalis hemagglutinin polypeptide, and wherein the fragment has anti-angiogenic activity.

32. The method of claim 31, wherein the polypeptide fragment is HA2.

33. A method of degrading a tumor comprising:
administering a pharmaceutically effective amount of a Porphyromonas gingivalis arginine specific cysteine protease polypeptide, Porphyromonas gingivalis lysine specific cysteine protease polypeptide, Porphyromonas gingivalis hemagglutinin polypeptide, or a combination thereof to a patient having a tumor, wherein the polypeptide degrades cell-cell bonds, cell-matrix bonds, or both cell-cell bonds and cell-matrix bonds of the tumor, whereby the tumor is degraded.

34. The method of claim 33 wherein the polypeptide is selected from the group consisting of rgpA, rgpB, kgp, hag, prtT and tla polypeptides.

35. The method of claim 34 wherein the polypeptide has at least about 90% se ce identity with a rgpA, rgpB, kgp, hag, prtT or tla polypeptide and wherein the polypeptide has anti-angiogenic activity.

36. The method of claim 33 wherein the polypeptide is a fragment of a Porphyromonas gingivalis arginine specific cysteine protease polypeptide, Porphyromonas gingivalis lysine specific cysteine protease polypeptide, or Porphyromonas gingivalis is hemagglutinin polypeptide, and wherein the fragment has anti-angiogenic activity.

37. The method of claim 36 wherein the polypeptide fragment is HA2.
